(19) United States
(12) Patent Application Publication
(10) Pub. No.: US 2026/0094671 A1
(43) Pub. Date: Apr. 2, 2026

(54) METHOD FOR PREDICTING STRUCTURE OF COMPOUND MODEL, METHOD FOR TRAINING MODEL, AND RELATED APPARATUSES

(71) Applicant: BEIJING BAIDU NETCOM SCIENCE TECHNOLOGY CO., LTD., Beijing (CN)

(72) Inventors: Lihang Liu, Beijing (CN); Yang Liu, Beijing (CN); Xianbin Ye, Beijing (CN); Shanzhuo Zhang, Beijing (CN); Xiaomin Fang, Beijing (CN); Xiaonan Zhang, Beijing (CN); Jingzhou He, Beijing (CN)

(73) Assignee: BEIJING BAIDU NETCOM SCIENCE TECHNOLOGY CO., LTD., Beijing (CN)

(21) Appl. No.: 19/413,463

(22) Filed: Dec. 9, 2025

(30) Foreign Application Priority Data
Jun. 3, 2025 (CN) 202510732969.6

(51) Int. Cl. G16B 30/00; G16B 5/20; G16B 40/20

(52) U.S. Cl. CPC G16B 30/00; G16B 5/20; G16B 40/20

(57) ABSTRACT

A method for predicting a structure of a compound includes: obtaining a combination of biomolecular sequences by combining specified biomolecular sequences; predicting a first probability distribution for the combination of biomolecular sequences, in which the first probability distribution is used for indicating first probabilities of candidate structural unit groups in the combination of biomolecular sequences, a candidate structural unit group includes structural units of at least two biomolecular sequences, and a first probability is used for indicating a possibility of interaction between structural units in a respective candidate structural unit group; determining, from the plurality of candidate structural unit groups, at least one first structural unit group based on the first probability distribution; and predicting a target structure of a biomolecular compound based on structural units interacted with each other in the at least one first structural unit group.

### CROSS-REFERENCE TO RELATED APPLICATIONS

The present application is based on and claims the priority of Chinese patent application No. 2025107329696 filed on Jun. 3, 2025, the entire content of which is incorporated herein by reference.

### TECHNICAL FIELD

The disclosure relates to the field of artificial intelligence technologies, in particular to the field of deep learning and biological computing technologies, and more particularly, to a method for predicting a structure of a compound, a method for training a model, and related apparatuses.

### BACKGROUND

With the rapid development of computer technology and bioinformatics, a biomolecular compound, as a basic unit for performing key biological functions, plays an irreplaceable role. In biology, multiple biomolecular sequences (such as proteins, nucleic acids and the like) form a compound through precise and dynamic interactions to realize core life activities such as signal transduction, gene regulation, metabolic catalysis and the like.

### SUMMARY

According to an aspect of the disclosure, there is provided a method for predicting a structure of a compound, including: obtaining a combination of biomolecular sequences, in which the combination of biomolecular sequences is obtained by performing a sequence combination based on multiple specified biomolecular sequences; predicting a first probability distribution for the combination of biomolecular sequences, in which the first probability distribution is used for indicating first probabilities of multiple candidate structural unit groups in the combination of biomolecular sequences, a candidate structural unit group includes structural units of at least two biomolecular sequences, and a first probability is used for indicating a possibility of interaction between structural units in a respective candidate structural unit group; determining, from the multiple candidate structural unit groups, at least one first structural unit group based on the first probability distribution; and predicting a target structure of a biomolecular compound based on structural units interacted with each other in the at least one first structural unit group.

According to another aspect of the disclosure, there is provided a method for training a structure prediction model, including: obtaining a training sample, in which the training sample includes a combination of sample biomolecular sequences, and the combination of sample biomolecular sequences is obtained by combining multiple sample biomolecular sequences; predicting a second probability distribution for the combination of sample biomolecular sequences using the structure prediction model, and generating a predicted structure of a biomolecular compound based on the second probability distribution, in which the second probability distribution is used for indicating second probabilities of multiple candidate structural unit groups in the combination of sample biomolecular sequences, a candidate structural unit group includes structural units of at least two sample biomolecular sequences, and a second probability is used for indicating a possibility of interaction between structural units in a respective candidate structural unit group; and training the structure prediction model based on a difference between a labeled structure and the predicted structure corresponding to the multiple sample biomolecular sequences.

According to yet another aspect of the disclosure, there is provided an electronic device, including:
at least one processor; and
a memory communicatively connected to the at least one processor,
in which the at least one processor is configured to obtain a combination of biomolecular sequences, in which the combination of biomolecular sequences is obtained by performing a sequence combination based on multiple specified biomolecular sequences; predict a first probability distribution for the combination of biomolecular sequences, in which the first probability distribution is used for indicating first probabilities of multiple candidate structural unit groups in the combination of biomolecular sequences, a candidate structural unit group includes structural units of at least two biomolecular sequences, and a first probability is used for indicating a possibility of interaction between structural units in a respective candidate structural unit group; determine, from the multiple candidate structural unit groups, at least one first structural unit group based on the first probability distribution; and predict a target structure of a biomolecular compound based on structural units interacted with each other in the at least one first structural unit group.

### BRIEF DESCRIPTION OF THE DRAWINGS

The accompanying drawings are used for better understanding the solution and do not constitute a limitation of the disclosure.

FIG. 1 is a flowchart illustrating a method for predicting a structure of a compound according to a first embodiment of the disclosure.

FIG. 2 is a flowchart illustrating a method for predicting a structure of a compound according to a second embodiment of the disclosure.

FIG. 3 is a flowchart illustrating a method for predicting a structure of a compound according to a third embodiment of the disclosure.

FIG. 4 is a flowchart illustrating a method for predicting a structure of a compound according to a fourth embodiment of the disclosure.

FIG. 5 is a flowchart illustrating a method for training a structure prediction model according to a fifth embodiment of the disclosure.

FIG. 6 is a schematic diagram illustrating a principle of a method for training a structure prediction model according to embodiments of the disclosure.

FIG. 7 is a schematic diagram illustrating a structure of an apparatus for predicting a structure of a compound according to a sixth embodiment of the disclosure.

FIG. 8 is a schematic diagram illustrating a structure of an apparatus for training a structure prediction model according to a seventh embodiment of the disclosure.

FIG. 9 is a block diagram illustrating an electronic device according to embodiments of the disclosure.

### DETAILED DESCRIPTION

Description will be made below to embodiments of the disclosure with reference to accompanying drawings, which includes various details of embodiments of the disclosure to facilitate understanding and should be regarded as merely examples. Therefore, it should be recognized by the skilled in the art that various changes and modifications may be made to the embodiments described herein without departing from the scope and spirit of the disclosure. Meanwhile, for clarity and conciseness, descriptions for well-known functions and structures are omitted in the following description.

In recent years, with the rapid development of artificial intelligence (AI) technology, AI-based structural prediction methods of biomolecular compounds (including proteins, small molecules, Ribonucleic Acid (RNA), Deoxyribonucleic Acid (DNA) and others) have attracted widespread attention from academia and industry compared with traditional experimental technologies. How to improve the accuracy and efficiency of structure prediction of biomolecular compounds with the help of deep learning technology has become the core topic of current research. Its application scenarios are wide, including but not limited to three-dimensional structure prediction of protein, interaction conformation prediction between protein and small molecule, RNA structure prediction and the like. Given biomolecular sequences (such as protein sequences, RNA sequences, DNA sequences), molecular formulas of small molecules, covalent information, protein modification information and the like, it is needed to predict the atomic-level 3D spatial structure of these biomolecules, including their own structure and binding posture. If an accurate structure may be obtained, it will play an important role for biologists to analyze biological processes and downstream applications, such as affinity prediction, drug screening, drug design and the like.

With the rapid development of computer technology and bioinformatics, a biomolecular compound, as a basic unit for performing key biological functions, plays an irreplaceable role. In biology, multiple biomolecular sequences (such as proteins, nucleic acids and the like) form a compound through precise and dynamic interactions to realize core life activities such as signal transduction, gene regulation, metabolic catalysis and the like. However, due to the high diversity of biomolecular sequences and the complexity of their interactions, accurately predicting and designing a three-dimensional structure of the biomolecular compound is still one of the most challenging problems in the current fields of computational biology and structural biology.

In the related art, an all-atomic structure prediction model is used to predict the structure of biomolecular compounds, in which the all-atomic structure prediction model (such as AlphaFold3) usually includes two networks: a structure generating network and a structure scoring network, in which the structure generating network is used to generate the corresponding conformation based on the inputted biomolecular sequence information, and the structure scoring network is used to score the generated conformation to assess the quality of the generated conformation. In the reasoning process of generated compound, the structure generating network will be called multiple times to generate different conformations, and then the structure scoring network will be used to score these conformations. The conformation with a highest score will be selected as the final predicted conformation. In order to make different generated conformations to have difference from each other, the following methods are usually relied on:
(1) disturbing data the input, such as disturbing multiple sequence alignment (MSA) information in the input;
(2) disturbing the network structure, such as activating a random dropout module of the network;
(3) a structure generating module based on a diffusion framework, its output itself being random. Different diffusion random seeds may lead to different conformations.

Although related sampling techniques may provide different conformations, their sampling is extremely random. For example, highly similar conformations may be sampled repeatedly, or it is difficult to sample the correct conformation, resulting in low efficiency.

In view of at least one of the above-described problems, the disclosure provides a method for predicting a structure of a compound, a method for training a model, and related apparatuses.

Hereinafter, a method for predicting a structure of a compound, a method for training a model, and related apparatuses according to embodiments of the disclosure will be described with reference to the drawings.

FIG. 1 is a flow chart illustrating a method for predicting a structure of a compound according to a first embodiment of the disclosure.

Embodiments of the disclosure illustrates that the method for predicting the structure of the compound is performed by an apparatus for predicting the structure of the compound, and the apparatus for predicting the structure of the compound may be applied to any electronic device, such that the electronic device may perform a function for predicting the structure of the compound.

The electronic device may be any device having computing capabilities, for example, a computer, a mobile terminal, a server and the like, and the mobile terminal may be, for example, a hardware device having various operating systems, touch screens, and/or displays, such as a vehicle-mounted device, a mobile phone, a tablet computer, a personal digital assistant, or a wearable device.

As illsutrated in FIG. 1, the method for predicting the structure of the compound may include the following.

At block 101, a combination of biomolecular sequences is obtained.

The combination of biomolecular sequences is obtained by performing a sequence combination based on multiple specified biomolecular sequences.

In order to effectively simulate potential interaction patterns between different biomolecular sequences, in embodiments of the disclosure, multiple specified biomolecular sequences are paired or combined together in different ways in order to further analyze possible interactions between different biomolecular sequences. The multiple specified biomolecular sequences include, for example, a protein sequence and a nucleic acid sequence, or include, for example, two or more different protein sequences.

At block 102, a first probability distribution for the combination of biomolecular sequences is predicted.

The first probability distribution is configured to indicate first probabilities of multiple candidate structural unit groups in the combination of biomolecular sequences. A candidate structural unit group includes structural units of at least two biomolecular sequences, and a first probability is used for indicating a possibility of interaction between structural units in a respective candidate structural unit group.

It should be understood that each of the biomolecular sequences may include multiple structural units (also referred to as tokens). For example, for protein sequences, DNA sequences, and RNA sequences, each residue is regarded as a token. In embodiments of the disclosure, for each combination of biomolecular sequences, a respective probability distribution (also referred to as the first probability distribution) is further predicted to evaluate which structural units in the combination of biomolecular sequences are most likely to interact with each other.

At block 103, at least one first structural unit group is determined from the multiple candidate structural unit groups based on the first probability distribution.

In order to accurately determine a structural unit group in which structural units are most likely to interact with each other, from the multiple candidate structural unit groups in the combination of biomolecular sequences, as a possible implementation, the at least one first structural unit group is determined from the multiple candidate structural unit groups based on the first probabilities, indicated by the first probability distribution, of the multiple candidate structural unit groups in the combination of biomolecular sequences.

At block 104, a target structure of a biomolecular compound is predicted based on structural units interacted with each other in the at least one first structural unit group.

Further, the target structure of the biomolecular compound is predicted based on relevant information of the first structural unit group. The relevant information of the first structural unit group includes, but is not limited to, detailed information such as positions, types, and an interaction mode of the structural units.

For example, if the first structural unit group includes specific amino acid residues (e.g., lysine and aspartic acid) and it is known that there may be non-covalent interactions between them (e.g., salt bridging), such interactions will be specifically taken into account in a case of predicting the structure of the compound.

In conclusion, the combination of biomolecular sequences is obtained by combining specified biomolecular sequences, potential interaction patterns between different biomolecules are simulated, and thus a rich candidate space for subsequent structure prediction is provided. Further, the possibility of occurrence of the interaction between structural units in the respective candidate structural unit group in the combination of biomolecular sequences is quantitatively evaluated based on the predicted first probability distribution of the combination of biomolecular sequences, to select the first structural unit group in which structural units are most likely to interact with each other, thereby avoiding a blind search of massive conformation space. Finally, the overall three-dimensional structure of the target biomolecular compound is further predicted based on structural unit information of structural units interacted with each other in the at least one first structural unit group. In this way, it is ensured that the structure of the generated biomolecular compound may meet the interaction requirements based on the structural units interacted with each other in the first structural unit group, thereby improving the reliability and biological rationality of predicting the structure.

For clearly explaining how the target structure of the biomolecular compound is predicted based on the structural units interacted with each other in the at least one first structural unit group in any embodiment of the disclosure, the disclosure provides another method for predicting the structure of the compound.

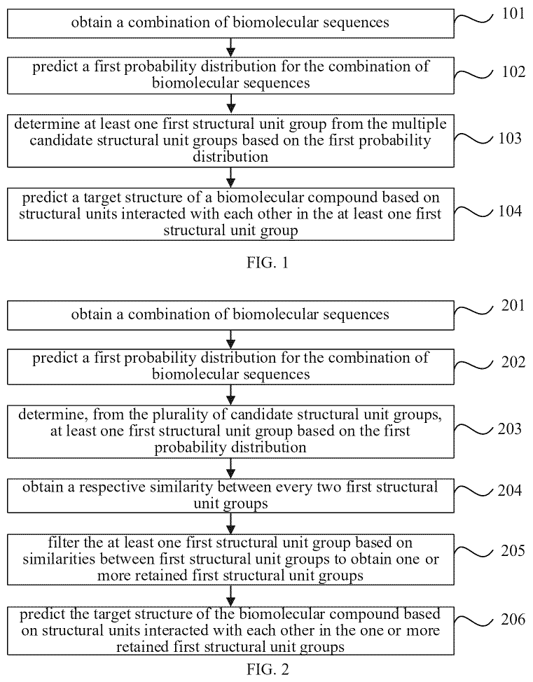

FIG. 2 is a flowchart illustrating a method for predicting a structure of a compound according to a second embodiment of the disclosure.

As illustrated in FIG. 2, the method for predicting the structure of the compound may include the following.

At block 201, a combination of biomolecular sequences is obtained.

The combination of biomolecular sequences is obtained by performing a sequence combination based on multiple specified biomolecular sequences.

At block 202, a first probability distribution for the combination of biomolecular sequences is predicted.

The first probability distribution is used for indicating first probabilities of multiple candidate structural unit groups in the combination of biomolecular sequences. A candidate structural unit group includes structural units of at least two biomolecular sequences, and a first probability is used for indicating a possibility of interaction between structural units in a respective candidate structural unit group.

At block 203, at least one first structural unit group is determined from the multiple candidate structural unit groups based on the first probability distribution.

In order to accurately select a structural unit group in which structural units are most likely to interact with each other, as a possible implementation, the first structural unit group of which structural units are most likely to interact with each other is determined from the candidate structural unit groups based on the first probabilities, in the first probability distribution, of the respective candidate structural unit group.

As an example, the at least one first structural unit group is determined from the multiple candidate structural unit groups based on the respective first probability of each candidate structural unit group in the first probability distribution, in which a first probability of each first structural unit group is greater than a preset threshold.

That is, a threshold is set as a standard for selection, and only the candidate structural unit group whose first probability exceeds the threshold is selected as the first structural unit group, which ensures that the selected first structural unit group has high reliability and stability to effectively excludes structural unit groups with low probability or instability, and thus improves the accuracy and reliability of predicting the structure of the biomolecular compound.

At block 204, a respective similarity between every two first structural unit groups is obtained.

A similarity is used for indicating a similarity degree between biomolecular compounds generated from structural units interacted with each other in respective two first structural unit groups.

In order to avoid from repeatedly sampling highly similar conformations, as a possible implementation, the respective similarity between every two first structural unit groups is determined, and the first structural unit group is selected based on the respective similarity between every two first structural unit groups. Therefore, the respective similarity between every two first structural unit groups may be obtained first.

As an example, a structure prediction is performed based on every two first structural unit groups to obtain predicted structures of every two first structural unit groups, in which a predicted structure is used for indicating a structure of the biomolecular compound generated based on a respective first structural unit group; a respective similarity degree between structures of the biomolecular compound generated by every two first structural unit groups is evaluated, based on the predicted structures of every two first structural unit groups; and the respective similarity between every two first structural unit groups is determined based on the respective similarity degree.

That is, for each pair of selected first structural unit groups (that is, a combination including at least two structural units), a computational biology tool or an algorithm are used for predicting the overall three-dimensional structures (i.e., the predicted structures) of the biomolecular compound that are formed from the pair of selected first structural unit groups, and then a comparison of the spatial overlap, atomic position difference and other geometric and topological characteristics is performed between the two predicted structures to determine the similarity score of the two predicted structures. The similarity score is used for indicating a respective similarity degree between structures of the biomolecular compound generated by any two first structural unit groups. Based on the similarity score, the similarity between any two first structural unit groups is determined. It should be noted that the higher the respective similarity degree between structures of the biomolecular compound generated by any two first structural unit groups, the higher the similarity between the two first structural unit groups.

At block 205, the at least one first structural unit group is filtered based on similarities between first structural unit groups to obtain one or more retained first structural unit groups.

In order to remove highly similar structural unit groups, reduce unnecessary repeated calculations, and save time and computing resources, as a possible implementation, the first structural unit group is filtered based on a set similarity threshold. If certain first structural unit groups are very similar to each other (i.e. the similarity exceeds a set threshold), a representative first structural unit group may be selected therefrom and retained and other similar first structural unit groups are removed, thereby obtaining the retained first structural unit groups.

At block 206, the target structure of the biomolecular compound is predicted based on structural units interacted with each other in the one or more retained first structural unit groups.

It should be understood that since the retained first structural unit groups represent different interaction patterns and structural features, in order to improve the diversity of predicted structures of the biomolecular compound, the target structure of the biomolecular compound is predicted based on relevant information of the structural units interacted with each other in the retained first structural unit groups.

It should be noted that the execution process of steps 201 to 202 may be implemented by any one of the embodiments of the disclosure, which is not limited in the embodiments of the disclosure and will not be repeated here.

In conclusion, the respective similarity between every two first structural unit groups is calculated, and all first structural unit groups are filtered based on the similarities, such that highly similar or repeated first structural unit groups may be effectively removed, to retain more representative and diverse first structural unit groups. This process not only reduces redundant calculations in subsequent structure prediction, improves computational efficiency, but also avoids the problem of falling into local optimal or repeated sampling of similar conformations. Furthermore, the target structure of the biomolecular compound is predicted based on the structural units interacted with each other in the one or more retained first structural unit groups, which is helpful to focus on the key interaction mode and improve the accuracy and biological rationality of the prediction results.

For clearly explaining how the target structure of the biomolecular compound is predicted based on structural units interacted with each other in the retained first structural unit groups in any embodiment of the disclosure, the disclosure provides another method for predicting the structure of the compound.

FIG. 3 is a flowchart illustrating a method for predicting a structure of a compound according to a third embodiment of the disclosure.

As illustrated in FIG. 3, the method for predicting the structure of the compound may include the following.

At block 301, a combination of biomolecular sequences is obtained.

The combination of biomolecular sequences is obtained by performing a sequence combination based on multiple specified biomolecular sequences.

At block 302, a first probability distribution for the combination of biomolecular sequences is predicted.

The first probability distribution is used for indicating first probabilities of multiple candidate structural unit groups in the combination of biomolecular sequences. A candidate structural unit group includes structural units of at least two biomolecular sequences, and a first probability is used for indicating a possibility of interaction between structural units in a respective candidate structural unit group.

At block 303, at least one first structural unit group is determined from the multiple candidate structural unit groups based on the first probability distribution.

At block 304, a respective similarity between every two first structural unit groups is obtained.

A similarity is used for indicating a similarity degree between biomolecular compounds generated from structural units interacted with each other in any two first structural unit groups.

At block 305, the at least one first structural unit group is filtered based on similarities between first structural unit groups to obtain one or more retained first structural unit groups.

At block 306, a respective candidate structure of the biomolecular compound corresponding to each retained first structural unit group is predicted based on the structural units interacted with each other in each retained first structural unit group.

In order to improve computational efficiency and diversity of results and avoid repeated sampling, as a possible implementation, multiple rounds of sampling are performed on each retained first structural unit group, one unlabeled structural unit group is selected and labelled from the retained first structural unit group in each round, and the corresponding structure of the biomolecular compound is generated based on the structural units interacted with each other in the sampled structural unit group.

As an example, multiple rounds of sampling are performed on each retained first structural unit group, in which each round of sampling includes: sampling one unlabeled first structural unit group from the retained first structural unit group and labeling the unlabeled first structural unit group with a label, the label is used for indicating that the first structural unit group has been sampled; and generating a candidate structure based on structural units interacted with each other in the sampled first structural unit group.

That is, in each round of sampling, one structural unit group is selected randomly or based on a certain strategy from the first structural unit groups that has not been labeled (i.e., has not been processed). Once a certain first structural unit group is selected, it will be labeled. The label is to indicate that the structural unit group has been sampled and processed, thereby avoiding repeated processing of the same structural unit group in the same round or in multiple rounds of sampling. For each sampled and labeled first structural unit group, the structure (that is, the candidate structure) of the biomolecular compound will be generated based on the structural units interacted with each other included therein.

At block 307, a respective structure score is predicted for each candidate structure.

The structure score is used for indicating a matching degree of the candidate structure with structural units of a respective first structural unit group corresponding to the candidate structure.

In order to effectively evaluate the rationality of each candidate structure and select the optimal conformation, as a possible implementation, the respective structure score is predicted for each candidate structure by evaluating the matching degree of each candidate structure of the biomolecular compound with the structural units of each first structural unit group corresponding to each candidate structure. For example, the structure score may be calculated with various methods, such as methods based on energy function, geometric matching degree, comparison of known similar structures, and the like. It should be noted that in a case where the structure score is higher, it is indicated that the candidate structure is more in line with the expected combination of structural units and their interaction mode.

At block 308, the target structure is determined from the candidate structures based on the respective structure score for each candidate structure.

In order to improve the prediction quality of the structure of the biomolecular compound, as a possible implementation, all candidate structures may be sorted based on their scores, and the candidate structure with a highest score may be directly selected as the only target structure.

It should be noted that the execution process of steps 301 to 305 may be implemented by any one of the embodiments of the disclosure, which is not limited in the embodiments of the disclosure and will not be repeated here.

In conclusion, the respective candidate structure of the biomolecular compound is predicted based on the structural units interacted with each other in each retained first structural unit group, such that a comprehensive exploration of potential compound conformation is achieved, thereby ensuring that all possible structural patterns are evaluated and avoiding missing any potentially important structure information. Further, the structure score is calculated for each candidate structure, the score indicating a matching degree and stability of the candidate structure with structural units of a respective first structural unit group corresponding to the candidate structure. Finally, the target structure is determined from the candidate structures based on the calculated structure scores, thereby improving the quality and accuracy of the structure prediction of the biomolecular compound.

For clearly explaining how the probability distribution of the combination of biomolecular sequences is predicted in any of the embodiments of the disclosure, the disclosure provides another method for predicting the structure of the compound.

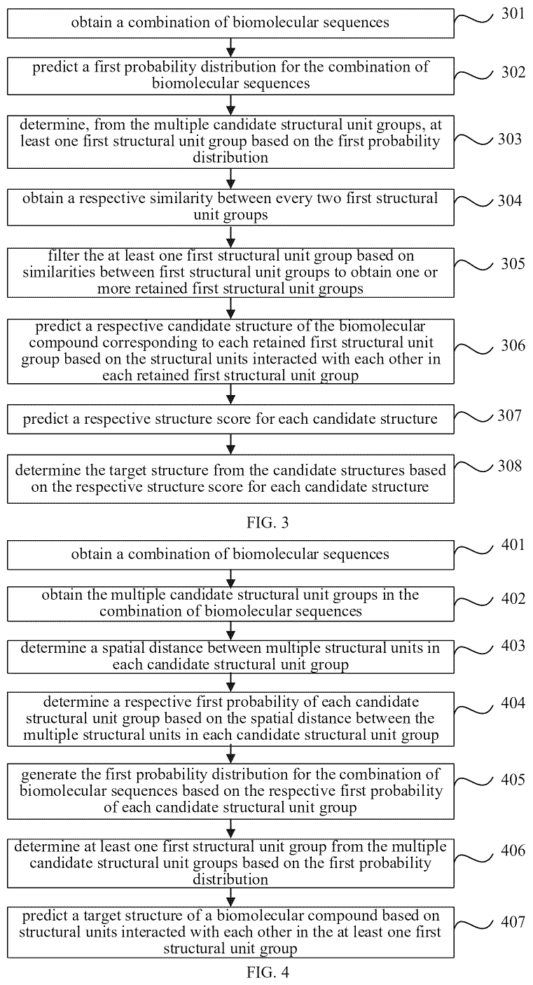

FIG. 4 is a flowchart illustrating a method for predicting a structure of a compound according to a fourth embodiment of the disclosure.

As illustrated in FIG. 4, the method for predicting the structure of the compound may include the following.

At block 401, a combination of biomolecular sequences is obtained.

The combination of biomolecular sequences is obtained by performing a sequence combination based on multiple specified biomolecular sequences.

At block 402, multiple candidate structural unit groups is obtained in the combination of biomolecular sequences.

In embodiments of the disclosure, the candidate structural unit group refers to a set of small molecule units that are likely to form a stable compound identified in the combination of biomolecular sequences based on certain criteria (such as physicochemical properties, known interaction databases and the like). Each candidate structural unit group includes at least two structural units (e.g. amino acid residues or nucleotides) that may interact with each other.

At block 403, a respective spatial distance between multiple structural units in each candidate structural unit group is determined.

In order to accurately determine whether the multiple structural units in each candidate structural unit group are physically “close” and thus have the potential of interaction, as a possible implementation, Euclidean distance formula and other methods are used to calculate the respective spatial distance between the multiple structural units in each candidate structural unit group.

At block 404, a respective first probability of each candidate structural unit group is determined based on the respective spatial distance between the multiple structural units in each candidate structural unit group.

The first probability is negatively correlated with the spatial distance.

As a possible implementation, the greater the spatial distance between the multiple structural units in a candidate structural unit group, the smaller the first probability of the candidate structural unit group, that is, the less the possibility of interaction between the structural units in the candidate structural unit group. The smaller the spatial distance between the multiple structural units in a candidate structural unit group, the greater the first probability of the candidate structural unit group, that is, the greater the possibility of interaction between the structural units in the candidate structural unit group.

At block 405, the first probability distribution is generated for the combination of biomolecular sequences based on the respective first probability of each candidate structural unit group.

Further, the first probabilities of all candidate structural unit groups are integrated into a comprehensive probability distribution map, that is, the first probability distribution for the combination of biomolecular sequences. The first probability distribution not only reflects the possibility of interaction (that is, the first probability) of each individual structural unit group, but also provides information about which structural unit groups in the combination of biomolecular sequences are more likely to form a stable compound.

At block 406, at least one first structural unit group is determined from the multiple candidate structural unit groups based on the first probability distribution.

At block 407, a target structure of a biomolecular compound is predicted based on structural units interacted with each other in the at least one first structural unit group.

It should be noted that the execution process of steps 401, 406 to 407 may be implemented by any one of the embodiments of the disclosure, which is not limited in the embodiments of the disclosure and will not be repeated here.

In conclusion, the respective spatial distance between the multiple structural units in each candidate structural unit group is calculated, and based on the respective spatial distance, the first probability of any candidate structural unit group is determined. Since the first probability is negatively correlated with the spatial distance, the smaller spatial distance corresponds to the higher first probability, which effectively quantifies the possibility of interaction between the structural units in the candidate structural unit group. Further, based on the respective first probability of each candidate structural unit group, the first probability distribution is generated for the combination of biomolecular sequences, which provides a basis for selecting first structural unit groups with high potentiality and improves the reliability and accuracy of structure prediction of the biomolecular sequences.

The above embodiments are embodiments corresponding to an application method for a structure prediction model (i.e. structure prediction of a compound), and the disclosure further provides a method for training a structure prediction model.

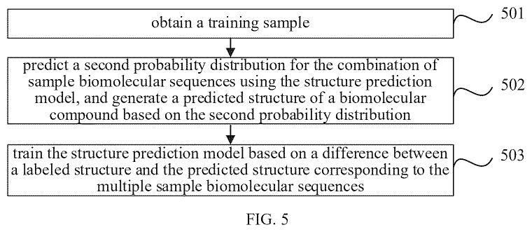

FIG. 5 is a flowchart illustrating a method for training a structure prediction model according to a fifth embodiment of the disclosure.

As illustrated in FIG. 5, the method for training the structure prediction model may include the following.

At block 501, a training sample is obtained.

The training sample includes a combination of sample biomolecular sequences, and the combination of sample biomolecular sequences is obtained by combining multiple sample biomolecular sequences.

As a possible implementation, the multiple sample biomolecular sequences are collected from a database, and the sample biomolecular sequences are combined to obtain the combination of sample biomolecular sequences. The training sample is generated based on the combination of sample biomolecular sequences. It should be noted that the training sample also includes a labeled structure of the compound corresponding to each combination of sample biomolecular sequences.

At block 502, a second probability distribution is predicted for the combination of sample biomolecular sequences using the structure prediction model, and a predicted structure of a biomolecular compound is generated based on the second probability distribution.

The second probability distribution is used for indicating second probabilities of multiple candidate structural unit groups in the combination of sample biomolecular sequences. A candidate structural unit group includes structural units of at least two sample biomolecular sequences, and a second probability is used for indicating a possibility of interaction between structural units in a respective candidate structural unit group.

As a possible implementation, the predicted combination of sample biomolecular sequences in the training sample is input into the structure prediction model, and the structure prediction model predicts the second probability distribution for the combination of sample biomolecular sequences and generates the predicted structure of the biomolecular compound based on the second probability distribution. The second probability distribution is used for indicating the second probabilities of the multiple candidate structural unit groups in the combination of sample biomolecular sequences, each candidate structural unit group includes the structural units of at least two sample biomolecular sequences, and the second probability is used for indicating the possibility of interaction between the structural units in the respective candidate structural unit group.

At block 503, the structure prediction model is trained based on a difference between a labeled structure and the predicted structure corresponding to the multiple sample biomolecular sequences.

In embodiments of the disclosure, a value of a loss function (referred to as a loss value in the disclosure) may be determined based on the difference between the labeled structure and the predicted structure, such that the structure prediction model may be trained based on the loss value by minimizing the loss value.

It should be noted that a termination condition of model training being minimizing the loss value is only an example. In practical application, other termination conditions may be set, for example, the termination conditions may also include: a training duration reaching a set duration, a training times reaching a set times and the like, which is not limited in embodiments of the disclosure.

In order to improve the accuracy and efficiency of structure prediction of the biomolecular compound, in any embodiment of the disclosure, the structure prediction model including a predicting network and a generating network is taken as an example, the second probability distribution for the combination of sample biomolecular sequences is predicted using the predicting network; at least one second structural unit group is determined from the multiple candidate structural unit groups using the generating network based on the second probability distribution; and the predicted structure of the biomolecular compound is generated using the generating network based on structural units interacted with each other in the at least one second structural unit group.

In order to improve the prediction accuracy of the structure prediction model, in any embodiment of the disclosure, at least one round of predicting is performed on the structural units interacted with each other in the at least one second structural unit group using the generating network. Each round of predicting includes: generating the predicted structure of the biomolecular compound based on structural units interacted with each other in one or more retained second structural unit groups. The one or more retained second structural unit groups are obtained by filtering the at least one second structural unit group based on a respective similarity between every two second structural unit groups.

In any embodiment of the disclosure, the structure prediction model further including a structure scoring network is taken as an example, generating the predicted structure of the biomolecular compound based on the structural units interacted with each other in one or more retained second structural unit groups mainly includes the following steps.
1. A candidate structure of the biomolecular compound corresponding to each retained second structural unit group is predicted using the generating network based on structural units interacted with each other in each retained second structural unit groups.

As an example, the multiple rounds of sampling are performed on each retained first structural unit group, in which each round of sampling includes: sampling one unlabeled first structural unit group from the retained first structural unit group and labeling the unlabeled first structural unit group with a label, in which the label is used for indicating that a respective first structural unit group has been sampled; and generating the candidate structure based on structural units interacted with each other in the one unlabeled first structural unit group.
2. The structure score of the candidate structure of the biomolecular compound corresponding to each retained second structural unit group is predicted using the structure scoring network, in which the structure score is used for indicating a matching degree of the candidate structure with structural units of a respective second structural unit group corresponding to the candidate structure.
3. A target structure is determined from the candidate structure of the biomolecular compound corresponding to each retained second structural unit group based on the structure score of the candidate structure of the biomolecular compound corresponding to each retained second structural unit group.

With the method for training the structure prediction model of the embodiments of the disclosure, the training sample including the combination of sample biomolecular sequences is obtained, in which the combination of sample biomolecular sequences is generated by combining the multiple sample biomolecular sequences, such that abundant input data is provided for the structure prediction model. Moreover, the second probability distribution is predicted for the combination of sample biomolecular sequences using the structure prediction model, and the predicted structure of the biomolecular compound is generated based on the second probability distribution, in which the second probability distribution quantifies the possibility of interaction between structural units in each structural unit group to be selected, and the structural unit group with the most possibility of interaction may be selected based on the second probability distribution. Furthermore, the three-dimensional structure of the biomolecular compound may be further predicted, thereby avoiding the blind search of the massive conformation space, and ensuring that the structure of the biomolecular compound generated based on the structural units interacted with each other in the structural unit group meets its interaction requirements. Finally, the structure prediction model is optimized and trained based on the difference between the known labeled structure and the predicted structure generated by the model corresponding to the multiple sample biomolecular sequences, thereby improving the accuracy and reliability of the model in predicting the structure of biomolecular compound.

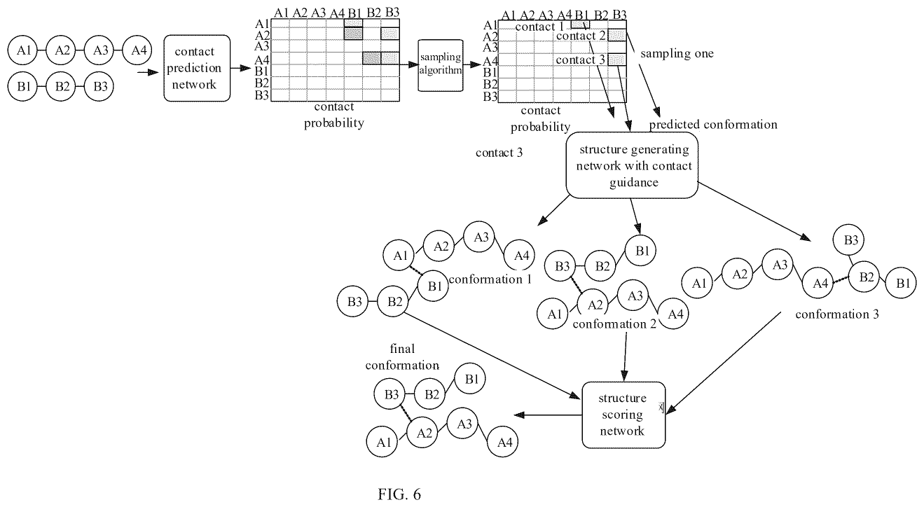

On the basis of any of the above embodiments, as illustrated in FIG. 6, the structure prediction model is an all-atomic structure prediction model. The all-atomic structure prediction model, for example, includes a predicting network (also referred to as a contact prediction network), a generating network (also referred to as a structure generating network with contact guidance) and a structure scoring network (also referred to as a structure scoring network), and the method for predicting the structure of the compound in embodiments of the disclosure may further be implemented based on the following steps:
(1) obtaining a combination of biomolecular sequences, in which the combination of biomolecular sequences includes a biomolecular sequence A (A1-A2-A3-A4) and a biomolecular sequence B (B1-B2-B3);
(2) predicting a probability distribution (also referred to as a contact probability distribution) for the combination of biomolecular sequences by using the predicting network, and identifying a group of contact sets (i.e., the first structural unit group) with a high probability based on the probability distribution; and
(3) performing sampling on retained contact sets with the high probability (i.e., the retained first structural unit groups), and generating a conformation meeting contacts. Since these contacts may correspond to similar combination patterns, contact sets with similar combination patterns are grouped, that is, the contact sets with the high probability are selected to obtain the retained contact sets with the high probability. The method includes the following steps:
1) sampling one contact from the retained contact sets with the high probability (i.e. performing sampling on one retained first structural unit group);
2) predicting the structure by the generating network, in which the model is to generate a conformation that conforms to an input contact;
3) resampling one contact that has not been sampled, and repeating the previous steps; and
4) performing sampling again on all the contact sets with the high probability in a case where all the contact sets with the high probability have been sampled and the sampling process is still going on.

Finally, all the prediction structures are sorted based on the scores given by the structure scoring network, and a structure with a highest score is selected as a final prediction result.

Corresponding to the method for predicting the structure of the compound based on the above embodiments illustrated in FIGS. 1-4, the disclosure also provides an apparatus for predicting a structure of a compound. Since the apparatus for predicting the structure of the compound corresponds to the method for predicting the structure of the compound based on the above embodiments illustrated in FIGS. 1-4, the implementations of the method for predicting the structure of the compound are also applicable to the apparatus for predicting the structure of the compound in embodiments of the disclosure, which is not elaborated in embodiments of the disclosure.

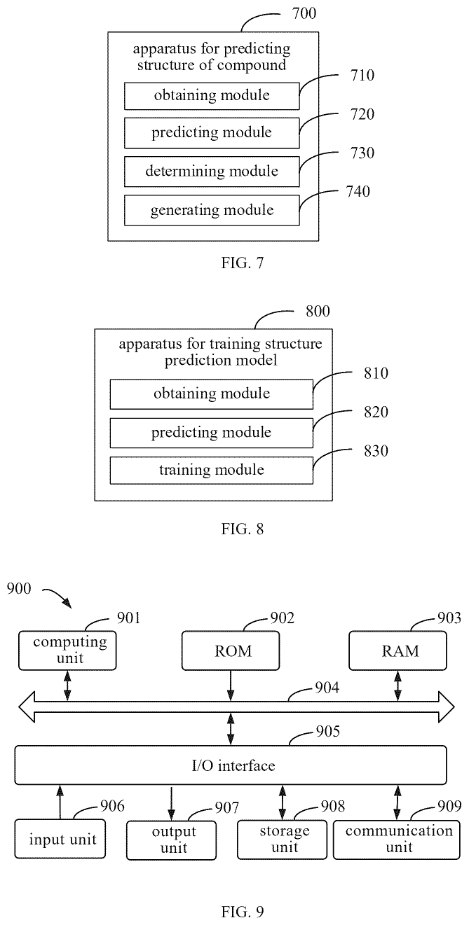

FIG. 7 is a schematic diagram illustrating a structure of an apparatus for predicting a structure of a compound according to a sixth embodiment of the disclosure.

As illustrated in FIG. 7, the apparatus 700 for predicting the structure of the compound may include: an obtaining module 710, a predicting module 720, a determining module 730, and a generating module 740.

The obtaining module 710 is configured to obtain a combination of biomolecular sequences, in which the combination of biomolecular sequences is obtained by performing a sequence combination based on multiple specified biomolecular sequences; the predicting module 720 is configured to predict a first probability distribution for the combination of biomolecular sequences, in which the first probability distribution is used for indicating first probabilities of multiple candidate structural unit groups in the combination of biomolecular sequences, a candidate structural unit group includes structural units of at least two biomolecular sequence, and a first probability is used for indicating a possibility of interaction between structural units in a respective candidate structural unit group; the determining module 730 is configured to determine, from the multiple candidate structural unit groups, at least one first structural unit group based on the first probability distribution; and the generating module 740 is configured to predict a target structure of a biomolecular compound based on structural units interacted with each other in the at least one first structural unit group.

As a possible implementation, the generating module 740 is configured to obtain a respective similarity between every two first structural unit groups, in which a similarity is used for indicating a similarity degree between biomolecular compounds generated from structural units interacted with each other in respective two first structural unit groups; filter the at least one first structural unit group based on similarities between first structural unit groups to obtain one or more retained first structural unit groups; and predict the target structure of the biomolecular compound based on structural units interacted with each other in the one or more retained first structural unit groups.

As a possible implementation, the generating module 740 is configured to predict a respective candidate structure of the biomolecular compound corresponding to each retained first structural unit group based on the structural units interacted with each other in each retained first structural unit group; predict a respective structure score for each candidate structure, in which the structure score is used for indicating a matching degree of the candidate structure with structural units of a respective first structural unit group corresponding to the candidate structure; and determine the target structure from the candidate structures based on the respective structure score for each candidate structure.

As a possible implementation, the generating module 740 is configured to perform multiple rounds of sampling on each retained first structural unit group, in which one round of sampling includes: sampling one unlabeled first structural unit group from the retained first structural unit group and labeling the unlabeled first structural unit group with a label, in which the label is used for indicating that a respective first structural unit group has been sampled; and generating a candidate structure based on structural units interacted with each other in the one unlabeled first structural unit group.

As a possible implementation, the generating module 740 is configured to perform a structure prediction based on every two first structural unit groups to obtain predicted structures of every two first structural unit groups, in which a predicted structure is used for indicating a structure of the biomolecular compound generated based on a respective first structural unit group; evaluate a respective similarity degree between structures of the biomolecular compound generated by every two first structural unit groups, based on the predicted structures of every two first structural unit groups; and determine the respective similarity between every two first structural unit groups based on the respective similarity degree.

As a possible implementation, the determining module 730 is configured to determine, from the multiple candidate structural unit groups, the at least one first structural unit group based on the respective first probability of each candidate structural unit group in the first probability distribution, in which a first probability of each first structural unit group is greater than a preset threshold.

As a possible implementation, the predicting module 720 is configured to obtain the multiple candidate structural unit groups in the combination of biomolecular sequences; determine a spatial distance between multiple structural units in each candidate structural unit group; determine a respective first probability of each candidate structural unit group based on the spatial distance between the multiple structural units in each candidate structural unit group, in which the first probability is negatively correlated with the spatial distance; and generate the first probability distribution for the combination of biomolecular sequences based on the respective first probability of each candidate structural unit group.

With the apparatus for predicting the structure of the compound, the combination of biomolecular sequences is obtained by combining the multiple specified biomolecular sequences, potential interaction patterns between different biomolecules are simulated, and thus a rich candidate space for subsequent structure prediction is provided. Further, the possibility of occurrence of the interaction between structural units in the respective candidate structural unit group in the combination of biomolecular sequences is quantitatively evaluated based on the predicted first probability distribution of the combination of biomolecular sequences, to select the first structural unit group in which structural units are most likely to interact with each other, thereby avoiding a blind search of massive conformation space. Finally, the overall three-dimensional structure of the target biomolecular compound is further predicted based on structural unit information of the structural units interacted with each other in the at least one first structural unit group. In this way, it is ensured that the structure of the generated biomolecular compound may meet the interaction requirements based on the structural units interacted with each other in the first structural unit group, thereby improving the reliability and biological rationality for predicting the structure.

Corresponding to the method for predicting the structure of the compound based on the above embodiments illustrated in FIGS. 5-6, the disclosure also provides an apparatus for predicting a structure of a compound. Since the apparatus for predicting the structure of the compound corresponds to the method for predicting the structure of the compound based on the above embodiments illustrated in FIGS. 5-6, the implementations of the method for predicting the structure of the compound are also applicable to the apparatus for predicting the structure of the compound in embodiments of the disclosure, which is not elaborated in embodiments of the disclosure.

FIG. 8 is a schematic diagram illustrating a structure of an apparatus for training a structure prediction model according to a seventh embodiment of the disclosure.

As illustrated in FIG. 8, the apparatus 800 for predicting the structure of the compound may include: an obtaining module 810, a predicting module 820, and a training module 830.

The obtaining module 810 is configured to obtain a training sample, in which the training sample includes a combination of sample biomolecular sequences, and the combination of sample biomolecular sequences is obtained by combining multiple sample biomolecular sequences; the predicting module 820 is configured to predict a second probability distribution for the combination of sample biomolecular sequences using the structure prediction model, and to generate a predicted structure of a biomolecular compound based on the second probability distribution, in which the second probability distribution is used for indicating second probabilities of multiple candidate structural unit groups in the combination of sample biomolecular sequences, a candidate structural unit group includes structural units of at least two sample biomolecular sequences, and a second probability is used for indicating a possibility of interaction between structural units in a respective candidate structural unit group; and the training module 830 is configured to train the structure prediction model based on a difference between a labeled structure and the predicted structure corresponding to the multiple sample biomolecular sequences.

As a possible implementation, the structure prediction model includes a predicting network and a generating network, and the predicting module 820 is configured to predict, using the predicting network, the second probability distribution for the combination of sample biomolecular sequences; determine, using the generating network, at least one second structural unit group from the multiple candidate structural unit groups based on the second probability distribution; and generate, using the generating network, the predicted structure of the biomolecular compound based on structural units interacted with each other in the at least one second structural unit group.

As a possible implementation, the predicting module is configured to perform, using the generating network, at least one round of predicting on the structural units interacted with each other in the at least one second structural unit group, in which one round of predicting includes: generating the predicted structure of the biomolecular compound based on structural units interacted with each other in one or more retained second structural unit groups, in which the one or more retained second structural unit groups are obtained by filtering the at least one second structural unit group based on a respective similarity between every two second structural unit groups.

With the method for training the structure prediction model of the embodiments of the disclosure, the training sample including the combination of sample biomolecular sequences is obtained, in which the combination of sample biomolecular sequences is generated by combining the multiple sample biomolecular sequences, such that abundant input data is provided for the structure prediction model. Moreover, the second probability distribution is predicted for the combination of sample biomolecular sequences using the structure prediction model, and the predicted structure of the biomolecular compound is generated based on the second probability distribution, in which the second probability distribution quantifies the possibility of interaction between structural units in each structural unit group to be selected, and based on the second probability distribution, the structural unit group with the most possibility of interaction may be selected. Furthermore, the three-dimensional structure of the biomolecular compound may be further predicted, thereby avoiding the blind search of the massive conformation space, and ensuring that the structure of the biomolecular compound generated based on the structural units interacted with each other in the structural unit group meets its interaction requirements. Finally, the structure prediction model is optimized and trained based on the difference between the known labeled structure and the predicted structure generated by the model corresponding to the multiple sample biomolecular sequences, thereby improving the accuracy and reliability of the model in predicting the structure of biomolecular compound.

To achieve the above embodiments, the disclosure also provides an electronic device. The electronic device may include at least one processor and a memory. The memory is communicatively connected with the at least one processor. The memory is configured to store instructions executed by the at least one processor. The instructions are executed by the at least one processor to enable the at least one processor to execute the method for predicting the structure of the compound based on any of the above embodiments of the disclosure, or execute the method for training the structure prediction model based on any of the above embodiments of the disclosure.

To achieve the above embodiments, the disclosure also provides a non-transitory computer readable storage medium having computer instructions stored thereon. The computer instructions are configured to cause a computer to execute the method for predicting the structure of the compound based on any of the above embodiments of the disclosure, or execute the method for training the structure prediction model based on any of the above embodiments of the disclosure.

To achieve the above embodiments, the disclosure also provides a computer program product. The computer program product includes a computer program. The computer program is configured to implement the method for predicting the structure of the compound based on any of the above embodiments of the disclosure, or implement the method for training the structure prediction model based on any of the above embodiments of the disclosure when processed by a processor.

According to embodiments of the present disclosure, the disclosure further provides an electronic device, a readable storage medium and a computer program product.

FIG. 9 is a block diagram illustrating an exemplary electronic device for implementing embodiments of the disclosure. The electronic device aims to represent various forms of digital computers, such as a laptop computer, a desktop computer, a workstation, a personal digital assistant, a server, a blade server, a mainframe computer and other suitable computer. The electronic device may also represent various forms of mobile devices, such as personal digital processing, a cellular phone, a smart phone, a wearable device and other similar computing device. The components, connections and relationships of the components, and functions of the components illustrated herein are merely examples, and are not intended to limit the implementation of the disclosure described and/or claimed herein.

As illustrated in FIG. 9, the device 900 includes a computing unit 901. The computing unit 901 may perform various appropriate actions and processes based on a computer program stored in a read only memory (ROM) 902 or loaded from a storage unit 908 into a random access memory (RAM) 903. In the RAM 903, various programs and data required for the operation of the device 900 may also be stored. The computing unit 901, the ROM 902, and the RAM 903 are connected to each other via a bus 904. An input/output (I/O) interface 905 is also connected to the bus 904.

Multiple components in the device 900 are connected to the I/O interface 905. The multiple components include an input unit 906, such as a keyboard, and a mouse; an output unit 907, such as various types of displays and speakers; a storage unit 908, such as a magnetic disk, and an optical disk; and a communication unit 909, such as a network card, a modem, and a wireless communication transceiver. The communication unit 909 allows the device 900 to exchange information/data with other devices via a computer network such as the Internet and/or various telecommunication networks.

The computing unit 901 may be various general-purpose and/or special-purpose processing components with processing and computing capabilities. Some examples of the computing unit 901 include, but are not limited to, a central processing unit (CPU), a graphics processing unit (GPU), various dedicated artificial intelligence (AI) computing chips, various computing units running machine learning model algorithms, a digital signal processor (DSP), and any suitable processor, controller, microcontroller, etc. The computing unit 901 performs various methods and processes described above, such as the above method for predicting the structure of the compound or the above method for training the molecular structure prediction model. For example, in some embodiments, the above method for predicting the structure of the compound or the above method for training the structure prediction model may be implemented as a computer software program tangibly embodied in a machine-readable medium, such as the storage unit 908. In some embodiments, a part or all of the computer program may be loaded and/or installed on the device 900 via the ROM 902 and/or the communication unit 909. When the computer program is loaded into the RAM 903 and executed by the computing unit 901, one or more steps of the method for generating the bicyclic peptide or the method for training the molecular structure prediction model described above may be executed. Alternatively, in other embodiments, the computing unit 901 may be configured to perform the above model pre-training method by any other suitable means (for example, by means of firmware).

Various implementations of the systems and techniques described above herein may be implemented in a digital electronic circuit system, an integrated circuit system, a field programmable gate array (FPGA), an application specific integrated circuit (ASIC), an application specific standard product (ASSP), a system on a chip (SOC), a load programmable logic device (CPLD), computer hardware, firmware, software, and/or a combination thereof. These various implementations may include being implemented in one or more computer programs. The one or more computer programs may be executed and/or interpreted on a programmable system including at least one programmable processor. The programmable processor may be a special purpose or general purpose programmable processor and receive data and instructions from and transmit data and instructions to a storage system, at least one input device, and at least one output device.

The program codes for implementing the method of the disclosure may be written in any combination of one or more programming languages. These program codes may be provided to a processor or controller of a general purpose computer, a special purpose computer, or other programmable data processing apparatus, such that the program codes, when executed by the processor or controller, cause the functions/operations specified in the flow charts and/or block diagrams to be implemented. The program codes may be executed completely on the machine, partially on the machine, partially on the machine as an independent software package and partially on a remote machine or completely on a remote machine or server.

In the context of the disclosure, a machine-readable medium may be a tangible medium, which may contain or store a program for use by or in connection with an instruction execution system, an apparatus or a device. The machine-readable medium may be a machine-readable signal medium or a machine-readable storage medium. The machine-readable medium may include, but is not limited to, an electronic, magnetic, optical, electromagnetic, infrared, or semiconductor system, an apparatus, or a device, or any suitable combination of the above. More specific examples of the machine-readable storage medium may include one or more wire-based electrical connections, a portable computer disk, a hard disk, a RAM, a ROM, an erasable programmable read-only memory (EPROM or flash memory), an optical fiber, a portable compact disk read-only memory (CD-ROM), an optical storage device, a magnetic storage device, or any suitable combination of the above.

To provide interaction with a user, the system and technologies described herein may be implemented on a computer. The computer has a display device (such as, a CRT (cathode ray tube) or a LCD (liquid crystal display) monitor) for displaying information to the user, a keyboard and a pointing device (such as, a mouse or a trackball), through which the user may provide the input to the computer. Other types of devices may also be configured to provide interaction with the user. For example, the feedback provided to the user may be any form of sensory feedback (such as, visual feedback, moderationory feedback, or tactile feedback), and the input from the user may be received in any form (including acoustic input, voice input or tactile input).

The system and technologies described herein may be implemented in a computing system including a background component (such as, a data server), a computing system including a middleware component (such as, an application server), or a computing system including a front-end component (such as, a user computer having a graphical user interface or a web browser through which the user may interact with embodiments of the system and technologies described herein), or a computing system including any combination of such background component, the middleware components and the front-end component. Components of the system may be connected to each other via digital data communication in any form or medium (such as, a communication network). Examples of the communication network include a local area network (LAN), a wide area network (WAN), an Internet and a blockchain network.

The computer system may include a client and a server. The client and the server are generally remote from each other and generally interact via the communication network. A relationship between the client and the server is generated by computer programs operated on a corresponding computer and having a client-server relationship with each other. The server may be a cloud server, also known as a cloud computing server or a cloud host, which is a host product in a cloud computing service system, to solve difficult management and weak business scalability in conventional physical host and VPS (virtual private server) services. The server may also be a distributed system server or a server combined with a block chain.

It should be understood that, artificial intelligence is a subject that studies computers to simulate certain thought processes and intelligent behaviors (such as learning, reasoning, thought, and planning) of humans. The AI relates to both hardware and software technologies. The hardware technologies of the artificial intelligence generally include technologies such as sensors, dedicated artificial intelligence chips, cloud computing, distributed storage, and big data processing. The software technologies of the artificial intelligence include some aspects such as computer vision technologies, speech recognition technologies, natural language processing technologies and learning, big data processing technologies, and knowledge map technologies.

According to the technical solution of embodiments of the disclosure, the combination of biomolecular sequences is obtained by performing the sequence combination based on the multiple specified biomolecular sequences, potential interaction patterns between different biomolecules are simulated, and thus a rich candidate space for subsequent structure prediction is provided. Further, the possibility of occurrence of interaction between structural units in the respective candidate structural unit group in the combination of biomolecular sequences is quantitatively evaluated based on the predicted first probability distribution of the combination of biomolecular sequences, thereby selecting the first structural unit group in which structural units are most likely to interact with each other, and avoiding a blind search of massive conformation space. Finally, the overall three-dimensional structure of the target biomolecular compound is further predicted based on structural unit information of the structural units interacted with each other in the at least one first structural unit group. In this way, it is ensured that the structure of the generated biomolecular compound may meet the interaction requirements based on the structural units interacted with each other in the first structural unit group, thereby improving the reliability and biological rationality for predicting the structure.

It should be understood that, steps may be reordered, added or deleted by utilizing flows in the various forms illustrated above. For example, the steps described in the disclosure may be executed in parallel, sequentially or in different orders, so long as desired results of the technical solution disclosed in the disclosure may be achieved, there is no limitation here.

The above detailed implementations do not limit the protection scope of the disclosure. It should be understood by the skilled in the art that various modifications, combinations, sub-combinations and substitutions may be made based on design requirements and other factors. Any modification, equivalent substitution and improvement made within the principle of the disclosure shall be included in the protection scope of the disclosure.

What is claimed is:

1. A method for predicting a structure of a compound, comprising:
obtaining a combination of biomolecular sequences, wherein the combination of biomolecular sequences is obtained by performing a sequence combination based on a plurality of specified biomolecular sequences;
predicting a first probability distribution for the combination of biomolecular sequences, wherein the first probability distribution is used for indicating first probabilities of a plurality of candidate structural unit groups in the combination of biomolecular sequences, a candidate structural unit group comprises structural units of at least two biomolecular sequences, and a first probability is used for indicating a possibility of interaction between structural units in a respective candidate structural unit group;
determining, from the plurality of candidate structural unit groups, at least one first structural unit group based on the first probability distribution; and
predicting a target structure of a biomolecular compound based on structural units interacted with each other in the at least one first structural unit group.

2. The method of claim 1, wherein predicting the target structure of the biomolecular compound based on the structural units interacted with each other in the at least one first structural unit group comprises:
obtaining a respective similarity between every two first structural unit groups, wherein a similarity is used for indicating a similarity degree between biomolecular compounds generated from structural units interacted with each other in respective two first structural unit groups;
filtering the at least one first structural unit group based on similarities between first structural unit groups to obtain one or more retained first structural unit groups; and
predicting the target structure of the biomolecular compound based on structural units interacted with each other in the one or more retained first structural unit groups.

3. The method of claim 2, wherein predicting the target structure of the biomolecular compound based on the structural units interacted with each other in the one or more retained first structural unit groups comprises:
predicting a respective candidate structure of the biomolecular compound corresponding to each retained first structural unit group based on the structural units interacted with each other in each retained first structural unit group;
predicting a respective structure score for each candidate structure, wherein the structure score is used for indicating a matching degree of the candidate structure with structural units of a respective first structural unit group corresponding to the candidate structure; and
determining the target structure from the candidate structures based on the respective structure score for each candidate structure.

4. The method of claim 3, wherein predicting the respective candidate structure of the biomolecular compound corresponding to each retained first structural unit group based on the structural units interacted with each other in each retained first structural unit group comprises:
performing a plurality of rounds of sampling on each retained first structural unit group, wherein one round of sampling comprises:
sampling one unlabeled first structural unit group from the retained first structural unit group and labeling the unlabeled first structural unit group with a label, wherein the label is used for indicating that a respective first structural unit group has been sampled; and
generating a candidate structure based on structural units interacted with each other in the one unlabeled first structural unit group.

5. The method of claim 2, wherein obtaining the respective similarity between every two first structural unit groups comprises:
performing a structure prediction based on every two first structural unit groups to obtain predicted structures of every two first structural unit groups, wherein a predicted structure is used for indicating a structure of the biomolecular compound generated based on a respective first structural unit group;
evaluating a respective similarity degree between structures of the biomolecular compound generated by every two first structural unit groups, based on the predicted structures of every two first structural unit groups; and
determining the respective similarity between every two first structural unit groups based on the respective similarity degree.

6. The method of claim 1, wherein determining, from the plurality of candidate structural unit groups, the at least one first structural unit group based on the first probability distribution comprises:
determining, from the plurality of candidate structural unit groups, the at least one first structural unit group based on the respective first probability of each candidate structural unit group in the first probability distribution,
wherein a first probability of each first structural unit group is greater than a preset threshold.

7. The method of claim 1, wherein predicting the first probability distribution for the combination of biomolecular sequences comprises:
obtaining the plurality of candidate structural unit groups in the combination of biomolecular sequences;
determining a spatial distance between a plurality of structural units in each candidate structural unit group;
determining a respective first probability of each candidate structural unit group based on the spatial distance between the plurality of structural units in each candidate structural unit group, wherein the first probability is negatively correlated with the spatial distance; and
generating the first probability distribution for the combination of biomolecular sequences based on the respective first probability of each candidate structural unit group.

8. A method for training a structure prediction model, comprising:
obtaining a training sample, wherein the training sample comprises a combination of sample biomolecular sequences, and the combination of sample biomolecular sequences is obtained by combining a plurality of sample biomolecular sequences;
predicting a second probability distribution for the combination of sample biomolecular sequences using the structure prediction model, and generating a predicted structure of a biomolecular compound based on the second probability distribution, wherein the second probability distribution is used for indicating second probabilities of a plurality of candidate structural unit groups in the combination of sample biomolecular sequences, a candidate structural unit group comprises structural units of at least two sample biomolecular sequences, and a second probability is used for indicating a possibility of interaction between structural units in a respective candidate structural unit group; and
training the structure prediction model based on a difference between a labeled structure and the predicted structure corresponding to the plurality of sample biomolecular sequences.

9. The method of claim 8, wherein the structure prediction model comprises a predicting network and a generating network, a predicted biomolecular compound is generated by the structure prediction model by:
predicting, using the predicting network, the second probability distribution for the combination of sample biomolecular sequences;
determining, using the generating network, at least one second structural unit group from the plurality of candidate structural unit groups based on the second probability distribution; and
generating, using the generating network, the predicted structure of the biomolecular compound based on structural units interacted with each other in the at least one second structural unit group.

10. The method of claim 9, wherein generating, using the generating network, the predicted structure of the biomolecular compound based on the structural units interacted with each other in the at least one second structural unit group comprises:
performing, using the generating network, at least one round of predicting on the structural units interacted with each other in the at least one second structural unit group, wherein one round of predicting comprises:
generating the predicted structure of the biomolecular compound based on structural units interacted with each other in one or more retained second structural unit groups, wherein the one or more retained second structural unit groups are obtained by filtering the at least one second structural unit group based on a respective similarity between every two second structural unit groups.

11. An electronic device, comprising:
at least one processor; and
a memory communicatively connected to the at least one processor,
wherein the at least one processor is configured to:
obtain a combination of biomolecular sequences, wherein the combination of biomolecular sequences is obtained by performing a sequence combination based on a plurality of specified biomolecular sequences;
predict a first probability distribution for the combination of biomolecular sequences, wherein the first probability distribution is used for indicating first probabilities of a plurality of candidate structural unit groups in the combination of biomolecular sequences, a candidate structural unit group comprises structural units of at least two biomolecular sequences, and a first probability is used for indicating a possibility of interaction between structural units in a respective candidate structural unit group;
determine, from the plurality of candidate structural unit groups, at least one first structural unit group based on the first probability distribution; and
predict a target structure of a biomolecular compound based on structural units interacted with each other in the at least one first structural unit group.

12. The electronic device of claim 11, wherein the at least one processor is configured to:
obtain a respective similarity between every two first structural unit groups, wherein a similarity is used for indicating a similarity degree between biomolecular compounds generated from structural units interacted with each other in respective two first structural unit groups;
filter the at least one first structural unit group based on similarities between first structural unit groups to obtain one or more retained first structural unit groups; and
predict the target structure of the biomolecular compound based on structural units interacted with each other in the one or more retained first structural unit groups.

13. The electronic device of claim 12, wherein the at least one processor is configured to:
predict a respective candidate structure of the biomolecular compound corresponding to each retained first structural unit group based on the structural units interacted with each other in each retained first structural unit group;
predict a respective structure score for each candidate structure, wherein the structure score is used for indicating a matching degree of the candidate structure with structural units of a respective first structural unit group corresponding to the candidate structure; and
determine the target structure from the candidate structures based on the respective structure score for each candidate structure.

14. The electronic device of claim 13, wherein the at least one processor is configured to:
perform a plurality of rounds of sampling on each retained first structural unit group, wherein each round of sampling comprises:
sampling one unlabeled first structural unit group from the retained first structural unit group and labeling the unlabeled first structural unit group with a label, wherein the label is used for indicating that a respective first structural unit group has been sampled; and
generating a candidate structure based on structural units interacted with each other in the one unlabeled first structural unit group.

15. The electronic device of claim 12, wherein the at least one processor is configured to:
perform a structure prediction based on every two first structural unit groups to obtain predicted structures of every two first structural unit groups, wherein a predicted structure is used for indicating a structure of the biomolecular compound generated based on a respective first structural unit group;
evaluate a respective similarity degree between structures of the biomolecular compound generated by every two first structural unit groups, based on the predicted structures of every two first structural unit groups; and
determine the respective similarity between every two first structural unit groups based on the respective similarity degree.

16. The electronic device of claim 11, wherein the at least one processor is configured to:
determine, from the plurality of candidate structural unit groups, the at least one first structural unit group based on the respective first probability of each candidate structural unit group in the first probability distribution,
wherein a first probability of each first structural unit group is greater than a preset threshold.

17. The electronic device of claim 11, wherein the at least one processor is configured to
obtain the plurality of candidate structural unit groups in the combination of biomolecular sequences;
determine a spatial distance between a plurality of structural units in each candidate structural unit group;
determine a respective first probability of each candidate structural unit group based on the spatial distance between the plurality of structural units in each candidate structural unit group, wherein the first probability is negatively correlated with the spatial distance; and
generate the first probability distribution for the combination of biomolecular sequences based on the respective first probability of each candidate structural unit group.

18. An electronic device, comprising:
at least one processor; and
a memory communicatively connected to the at least one processor,
wherein the at least one processor is configured to perform the method of claim 8.

19. A non-transitory computer-readable storage medium having stored therein a computer instruction, wherein the computer instruction enables a computer to perform the method of claim 1.

20. A non-transitory computer-readable storage medium having stored therein a computer instruction, wherein the computer instruction enables a computer to perform the method of claim 8.
